(19) United States
(12) Patent Application Publication
(10) Pub. No.: US 2014/0220652 A1
(43) Pub. Date: Aug. 7, 2014

(54) Methods And Systems For Absorbing CO2 And Converting Same Into Gaseous Oxygen By Microorganisms

(71) Applicant: Marcelo Fabián González Machíi, Las Palmas de Gran Canaria (ES)

(72) Inventor: Marcelo Fabián González Machíi, Las Palmas de Gran Canaria (ES)

(73) Assignees: CYBEL HOLDING S.A., Luxembourg (LU); Marcelo Fabián GONZÃLEZ MACHÍN, Las Palmas de Gran Canaria (ES)

(21) Appl. No.: 14/129,457

(22) PCT Filed: Nov. 24, 2011
(86) PCT No.: PCT/ES2011/070813

§ 371 Date: Apr. 15, 2014

(30) Foreign Application Priority Data
Jun. 28, 2011 (ES) 201100745

(51) Int. Cl. B01D 53/84; C12P 3/00; C12P 5/02

(52) U.S. Cl. CPC B01D 53/84; C12P 5/023; C12P 3/00
USPC 435/167; 435/168; 435/292.1

(57) ABSTRACT

Methods and systems are described for the purification of contaminated air containing CO₂, converting it into O₂ by means of the use of microorganisms. Said methods and systems comprise the initial steps of capturing the air proceeding from a source of contaminated air containing CO₂, such as an industrial plant, and subsequent physical catalysis of said contaminated air, passing it across plates which partially fix the CO₂ in the form of calcium and/or magnesium carbonates. Subsequent to these steps the air is passed through fermenter tanks containing a culture comprising a biofamily of microorganisms and an organic inhibitor permitting maximum absorption of CO₂ and emission of O₂. By means of the methods and systems described the conversion of CO₂ into O₂ is achieved having an efficiency very superior to those of the prior art.

### FIELD OF THE INVENTION

The present invention relates to methods and systems for the absorption of CO₂ and the conversion thereof into gaseous oxygen by means of microorganisms according to the preamble of Claims 1 and 18.

### BACKGROUND OF THE INVENTION

The present invention relates to methods and systems for the absorption of CO₂ and the conversion thereof into gaseous oxygen (O₂) utilising therefor particular microorganisms, such as certain microalgae, spores and manure. Preferably, the CO₂ proceeds from industrial sources, as a result whereof the objective is achieved of reducing atmospheric pollution, generating as a consequence gaseous oxygen beneficial for the environment.

The present invention is based on the observation of the author of the present invention in the sense that, according to diverse publications, certain microorganisms, microalgal biomass, etc, may be utilised having the dual objective of simultaneously absorbing CO₂ and generating lipids employing solely the natural photoperiod and basing the entire invention on the Calvin cycle or photosynthesis. In such cases the cultivation of biomass based on the natural solar light or photosynthetic cycle may confer a certain assurance of success for the production of lipids, together with a satisfactory level of actual absorption of CO₂, being intimately related with the capacity of cells themselves to generate triglycerides, cellular division, and the specific location of the installation, generally based in open pools or ponds or in vertical tubing wherein CO₂ is expelled from the base thereof and wherein a few seconds following the injection thereof a large part of the gaseous mass escapes again to the atmosphere. Nevertheless, if the appropriate conditions could be brought about such that the microorganisms do not lose energy in generating triglycerides but the same is employed in the reproduction thereof leading to an increased rate of growth and reproduction, the absorption of CO₂ and consequent production of O₂ thereby would be substantially increased.

Thus, for example, in the prior art there exist systems based on the cultivation of microalgae for the production of biofuels. All thereof utilise, principally, a single species of microalgae (monoculture) having a very low yield, not permitting resolving the economic equation of the process, and work at temperatures of between 22° C. and 28° C., operation thereof being restricted to zones having hot climates. On the contrary, the systems and methods of the present invention may be made to function at temperatures lower than 18° C., in particular between 14 and 16° C., this having been achieved by acclimatising the microalgae species utilised to such climatological conditions. Moreover, in the systems and methods of the invention appropriate conditions may be selected such that the production of triglycerides is at a minor level or even marginal, substantially orienting the production of the microorganisms towards gaseous O₂.

Moreover, in systems of the prior art, CO₂, and above all industrial CO₂, having an effect on climate change that is becoming more evident every day, is habitually accompanied by other greenhouse gases which are not taken into account in said systems either, which same would severely damage any terrestrial or aquatic biomass cultivation irremediably, as a consequence whereof in the present invention the application thereof has been taken into account very much on an industrial scale, efficiently and consciously based on the multiple disciplines involved and the combination of systems and subsystems rendering it technically viable and scalable.

Utilisation of microalgae for the absorption of CO₂ and the substantial conversion thereof into O₂ has been known for a long time, thus, for example, patent documents U.S. Pat. No. 3,224,143 and U.S. Pat. No. 3,303,608 have already described the conversion of carbon dioxide into oxygen through the use of algae.

More recently, documents WO 92/00380 and U.S. Pat. No. 5,614,378 describe the conversion of CO₂ into O₂ by cyanobacteria when the same are irradiated with radiation having wavelengths between 400 and 700 nm. However, the systems described in said documents are designed for the use thereof in artificial hearts, as a consequence whereof they are not optimised for the production of O₂ on an industrial scale as are those of the present invention, and they lack many of the technical characteristics described in the present invention.

Methods and systems directed towards the conversion of CO₂ into O₂ have also been described in other patent publications, such as EP 0 874 043 A1, EP 0 935 991 A1 and WO 2005/001104 A1, wherein species of Spirulina platensis and of Chlorella vulgaris were utilised as microalgae, and JP 2009007178 A, wherein marine cyanobacteria of the genus Acaryochloris were utilised.

Nonetheless, none of said documents describe or suggest methods and systems optimised for the conversion of CO₂ into O₂ presented by the characteristics and advances of the methods and systems of the present invention which, in particular, exhibit notably increased efficiency over those described in the prior art, as shall be described below.

### SUMMARY OF THE INVENTION

The principal objective sought in the present invention is the conversion of CO₂ into O₂ as a means of providing practical solutions to the latest regulations, laws and measures intended to reduce the carbon footprint to palliate the effects of global warming.

For this purpose, technological solutions have been put into practice utilising species of microalgae different from those customarily utilised in microalgae cultivation systems intended for the obtainment of biofuels, as are the working conditions thereof. The microorganisms utilised in the present invention are different species of microalgae, bacteria and spores which, in perfect symbiosis, act in an efficient manner in the capture of carbon dioxide (CO₂) originating from industrial sources and which, with the possible assistance of tracers based on calcium silicates, convert it into oxygen (O₂). Through the provision of suitable conditions such that the microorganisms practically do not consume energy in generating triglycerides, said energy is employed by the microorganisms in the reproduction thereof, maintaining a maximum rate of growth, finally redounding in a notably increased rate of production of O₂.

A further aspect considered in the present invention is the physical space necessary for the implementation thereof on an industrial scale. The majority of systems require natural light as has already been explained; as a consequence, based on the simple equation of solar irradiation per cm² of surface area, the conclusion is reached that to have the same efficiency as the present invention the systems and methods of the prior art require very extensive land areas which, in the majority of industrial estates, is impracticable or at least economically unviable.

As a consequence, a first aspect of the invention is directed towards a method for purifying contaminated air containing CO₂ by means of the use of microalgae, comprising the steps of:
a) reception of the air containing CO₂ proceeding from a source of contaminated air;
b) physical catalysis, wherein the air containing CO₂ is passed across plates comprising calcium and/or magnesium salts whereon part of the CO₂ of the air becomes fixed in the form of calcium and/or magnesium carbonates;
   characterised in that the method moreover comprises the steps of:
c) fermentation, wherein the air proceeding from step b) is passed across a culture comprising a biofamily of microorganisms together with at least one organic inhibitor selected from the group consisting of an alcohol, a ketone or a carboxylic acid in any combination thereof, in which step at least part of the remaining CO₂ in the air becomes dissolved in the culture, and;
d) irradiation of the culture with light radiation of the visible light spectrum having a particular frequency, intensity and duration,
   such that photosynthesis is promoted in said microorganisms, causing a reduction in the CO₂ content in the culture through absorption and/or digestion of said CO₂ in the microorganisms and producing O₂.

A second aspect of the invention is directed towards a system for putting into practice the method for purifying contaminated air containing CO₂ through the use of microalgae, comprising the following elements:
a) reception system (1) receiving air containing CO₂ proceeding from a source of contaminated air;
b) plates (2) containing calcium and/or magnesium salts intended to fix part of the CO₂ of the air in the form of calcium and/or magnesium carbonates;
   characterised in that the method moreover comprises:
c) tanks (3) for fermentation of the air proceeding from the previous step containing a culture comprising a family of microorganisms together with at least one organic inhibitor selected from the group consisting of an alcohol, a ketone or a carboxylic acid in any combination thereof,
wherein such tanks comprise moreover a source of light radiation intended to irradiate the culture with light radiation having a particular frequency, intensify and duration.

The system will be preferably linear in the case wherein the owner thereof desires to generate oxygen gradually. In this manner, if 100% conversion into oxygen of the gases emitted is not desired from the outset, said value may be attained gradually.

### BRIEF DESCRIPTION OF THE FIGURES

FIG. 1 attached shows a general illustrative diagram of a preferred embodiment of the systems and methods of the present invention.

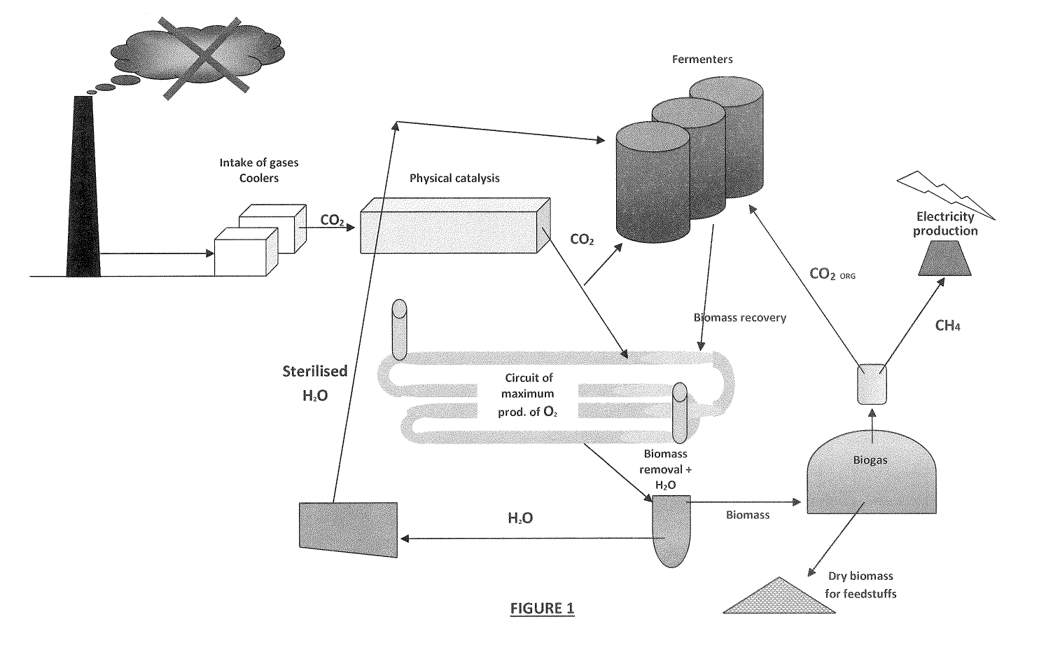

FIG. 2 shows the results obtained through the system described in Experimental Example no. 3.

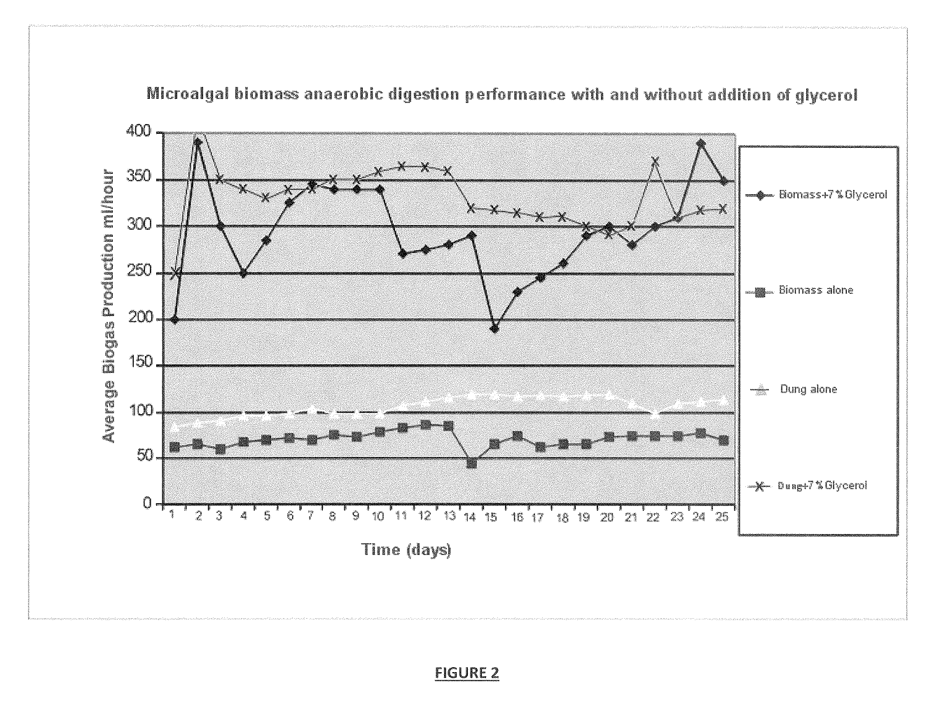

### DETAILED DESCRIPTION OF THE INVENTION

The present invention provides systems and methods for the absorption of CO₂, producing gaseous oxygen which may be emitted to the atmosphere, with the objective of reducing emissions of greenhouse gases, principally CO₂ together with methane gas, there moreover having been found the manner of so doing in the minimum space possible, based on artificial photosynthesis and other resources detailed below in the order of the flow diagram.

In a general manner, the culture is developed by providing the microorganisms with carbon dioxide (CO₂), which is mixed and partitioned, adding thereto macro and micro elements, water and tracer elements, being injected in a perfectly proportioned manner together with the CO₂ into blind photobioreactors irradiated with certain wavelengths of the visible spectrum delivered to the microorganisms in the form of photons having the required intensity to obtain a high degree of CO₂ absorption, inhibiting to a great extent the formation of triglycerides, which in other systems are intended for the production of biofuels, the majority being thus converted into O₂.

The systems and methods of the invention provide the possibility of the utilisation of fresh, brackish or salt water which, being utilised in closed circuit, prevents large losses of this resource being produced. Subsequent to taking part in the process the water may be sterilised for reutilisation thereof, to prevent any type of contamination, although the biosystem formed of multiple species of microorganisms substantially inherently prevents the development of other contaminant, competitive or destructive species.

In preferred embodiments, the sea, fresh or brackish water and the CO₂ are treated prior to entry thereof into the system of photobioreactors wherein they are incorporated with the microorganisms and the nutrients required such that, with the incidence of light, growth of the biomass of the system takes place.

The culture system utilised is mixotrophic by virtue of the fact that it is independent of natural light. The species have been acclimatised to receive artificial light of a particular wavelength and defined intensity such that maximum CO₂ capture occurs. Photoautotrophic systems, on the other hand, require natural light for growth of the culture to occur.

Having achieved the planned maximum growth of the biomass, the biomass resulting from the process is deactivated, extracting therefrom biogas through a fermentative oxidation process, utilising therefor natural biological triggers or inducers to accelerate the process (methanogenic bacteria). The composition of the biogas resulting from digestion of the biomass is of approximately 30-40% CO₂ and 60-70% methane. This methane gas (CH₄) obtained may be utilised, for example, for the generation of electricity by means of a turbine. In a particularly preferred embodiment, such turbine is a turbine of the author's design calculated for utilisation with methane (CH₄) and possessing a conversion efficiency of approximately 87%, differing from modified gasoil or fuel oil generators employing said gas having an approximate conversion efficiency of 50%. Finally, in preferred embodiments, both the calorific energy together with the low levels of emissions from this process are reutilised and reinjected into the system.

The systems and methods of the invention typically comprise the following steps:

1 Step of reception of gases: In general, the majority of large CO₂ emitters realise some type of mitigation of emissions of NOx, SOx and particles utilising diverse filters, catalysts and/or mechanical means, achieving differing degrees of mitigation of emissions, those of CO₂ being the most abundant, expensive and complex to mitigate by virtue of the volume thereof compared with other contaminants.

In a general manner, the present invention may include a reception or extraction system responsible for extracting or receiving the gases from the source of origin thereof and delivering them under conditions of solution, pressure, temperature and physical precatalysis appropriate for the remainder of the system. In one embodiment, in this reception step the invention possesses a cooling prechamber for the gases wherein they are received from the industrial installations of origin thereof through a simple conventional industrial extractor prepared for high temperatures. Should it be present, said extractor will be dimensioned in accordance with the volume of gas which the owner of each plant desires to be absorbed, and the precooling may be realised by any method known to a person skilled in the art.

In another particularly preferred embodiment, the reception system of gases comprises a circuit containing a particular solvent in circulation, wherein both the CO₂ and the other accompanying gases, such as NOx, SOx, etc, to mention some thereof, may be dissolved, buffered, precatalysed and/or cooled to the appropriate temperature, as may be deemed necessary or suitable, prior to entering the remainder of the system.

2 Step of physical catalysis: Having been appropriately cooled the gases proceeding from the foregoing step are passed to a step of physical catalysis. In a preferred embodiment this step is realised within a chamber having a humidity of from 40% to 90%, preferably 80% relative humidity, or a series of similar chambers wherein basically the cooled gases are made to circulate past catalytic plates placed in a horizontal or vertical orientation according to the space available. As will be seen below, the entire system may be located underground, principally by virtue of it being based on photosynthesis generated by artificial light, as a result whereof the visual pollution caused by systems found habitually in the market is moreover avoided.

Said plates have been designed and developed for the present invention starting with generally very abundant materials preferably originating from urban waste, principally calcium or magnesium silicates, bonding them in such manner that they present sufficient porosity to permit the passage of gases, particularly the gaseous CO₂ and the CO₂ dissolved in water. For this purpose, in a preferred embodiment, at the time of manufacture thereof and prior to the setting thereof, the plates are quickly pierced by needles of differing diameters, leaving therein a multiplicity of orifices piercing them diametrically, for example hundreds thereof, permitting an increase in the permeability thereof or, what is the same thing, the velocity of mineral carbonation in the same manner wherein CO₂ in nature reacts with non-carbonated minerals to form carbonates, which reactions in nature are normally slow; this constitutes a first barrier of secure fixing of CO₂ or oxygen generation. In a particularly preferred embodiment, said plates contain the following materials in the following proportions:
10% to 30% CaO;
5% to 25% calcium carbide (CaC₂);
15 to 25% calcium hydroxide Ca(OH)₂;
10 to 50% calcium carbonate (CaCO₃);
5% to 50% magnesium (Mg);
5% to 15% aluminium filings (Al).

In a particularly preferred embodiment, 50% of the aforementioned calcium carbonate is obtained from the finely ground shells of oysters, clams and scallops obtained from restaurants and seafood processing plants and, to a lesser degree, of those of mussels, which otherwise would end up in dumps, signifying a manner of increasing the value of waste which otherwise might become contaminants. The general reaction which occurs, as in nature, is exemplified in the following manner:

(Mg,Ca)ₓSi_{y}O_{x+2y+z}+xCO₂→x(Mg,Ca)CO₃+ySiO₂+zH₂O

Normally the percentage of finely ground calcium and magnesium silicates incorporated into the closed circuit of extraction of gases lies between 20 g/l and 250 g/l.

In particular locations, such as in Chubut, Argentina, wherefrom the inventor originates, high percentages of olivine have been found in local clayey soils which may also be utilised in different proportions in the aforementioned formulation, it having been observed that one tonne of pure olivine may achieve the storage (crushed) of up to 2.3 tonnes of CO₂ in a relatively short time.

Olivine from Chubut: Mg₂SiO₄+2CO₂→2MgCO₃+SiO₂

In the system described, and by virtue of the fact that normally sequestration of CO₂ from chimneys is undertaken through closed circuit scrubbing by water stream (physical catalysis module—chimney—physical catalysis module loop) also rich in silicates, consequently part of the fixing of CO₂ already occurs through the very dilution of the CO₂ in water and in permanent contact within this loop.

Said system, which furthermore may serve to cool the inlet gases which, in most plants, range between 130° C. and 600° C. in many cases, will generate steam pressure in the circuit and will raise the temperature in the first absorption module which will operate, depending on the plant in question in respect of the installation of this system, at a temperature of between 100 and 200° C. and under a regime which will increase the permeability of the plates and, as a consequence, the speed of CO₂ fixing. This module is made to work as a cascade condenser, balancing the pressures and extracting the CO₂ at a lower temperature by means of a vacuum towards a second optional physical catalysis module which will preferably work at lower temperatures, of the order of 21° C., and 1 atm pressure.

The original principal reactants (CO₂ and silicates) when mutually combined experience a reduction in the volume thereof, carbonates being some 900 times more dense (weighted average among those referred to) than CO₂ in gaseous state at approximately 20° C. and 1 atm. On the CO₂ being fixed in the minerals (solid phases) of the catalytic plates, that is to say the CO₂ having been incorporated into the solid phase, the assembly (depending on the mixtures and the percentages thereof) generates increases in weight and volume ranging from 10% to 150%. The panels, having attained saturation, may be exchanged and easily utilised by different types of plant, especially cement plants. Moreover, as already stated, the raw materials are abundant and may be obtained from different places at very competitive price and having become saturated they are 100% recyclable without any type of special treatment other than the grinding thereof once more.

Preferably, said plates are periodically analysed to evaluate the constitution and catalytic efficiency thereof. The most convenient standard dimensions of the module are 2.25 m wide×2.50 m high×12.5 m long in order that they may be rapidly exchanged by lorry and transported to special reassembly installations. Moreover, the new units may be interconnected such that it is not necessary to stop the cycle to change the plates of the integral oxygen production circuit. Diverse chemical reactions being realised in the catalysis, the containers therefor are preferably plastic coated internally and the floors thereof are installed in the form of trays to contain and recirculate the different elements of the plates which decompose and drip as a result of to the high humidity of the environment and acidification of the medium caused by the CO₂.

These modules will be preferably dimensioned to substantially absorb the remains of the NOx and SOx of the inlet gas, together with from 20% to 25% of the CO₂ contained therein, which the materials described in the composition of said plates will catalyse and/or convert into O₂. The 75 to 80% balance of CO₂ gas, although still containing minimal traces of NOx and SOx, will be sent to the subsequent fermentation step.

3 Fermentation step: The gases proceeding from the foregoing step are transferred to fermenter tanks wherein they are passed across a culture containing a biofamily of microorganisms of the type of especially acclimatised microalgae and spores having great capacity for the absorption or biofixation of CO₂ and liberation of oxygen. In a preferred embodiment, such biofamily of microorganisms comprises any subset of the following species:

Clorophyceae: Chlorella vulgaris, Chlorella saccharophila, Lobomonas sp, Scenedesmus acuminatus, Scenedesmus quadricauda, Scenedesmus sp, Scenedesmus desmodesmus, Ankistrodesmus angustus, Monoraphidium griffithii, Elakatothrix gelatinosa, Golenkinia radiata, Dictyosphaerium pulchellum, Sphaerocystis schroeteri, Oocystis sp, Selenodyctium brasiliensis,

Cyanophyceae: Chroococcus sp, Cyanophyceae filamentosa, Arthrospira platensis, Arthrospira maxima, Nostoc sp, Nostoc ellipsosporum, Nostoc spongiaeforme, Anabaena macrospora, Anabaena monticulosa, Anabaena azollae, Spirulina platensis, Spirulina maxima, Spirulina orovilca, Spirulina jeejibai, Spirulina lonar, Prorocentrum dentatum, Noctiluca scientillans, Trichodesmium sp., Aurantiochytrium,

Cryptophyceae: Cryptomonas sp, Cryptomonas brasiliensis;

Diatomaceae: Centrica (unidentified), Nitzschia sp, Skeletonema costatum,

Spores and manure: Brown Laminariales (Macrocystis pyrifera, Undaria pinnatifida); Red of Order Gigartinales (Gigartina skottsbergii, Kappaphycus alvarezil), Green of Order Ulvales (Enteromorpha prolifera).

All these species, in the form of an assembly thereof (biofamily) or separately or in any combinations and proportions of natural or induced predominance thereof, may be developed in fresh, brackish or sea water in appropriate proportions of dilution and temperature according to the region wherein the cultivation thereof is established, or they may be cultivated in water or in an artificial cultivation medium having the following characteristics, according to a preferred embodiment:

For every 1000 ml of distilled or bidistilled water there will be artificially added the following elements, ensuring very suitable performance of the species stated for the objective sought, the same being capable of being developed separately or as an assembly (biofamily), it being possible to regulate the proportions in the sense of the minimum percentages stated (less brackish to fresh water) or the maximum percentages stated (less brackish to sea water, including having salt lake characteristics):

NaCl From 3 to 33 g/l, preferably approximately 11 g/l;
KCl From 0.1 to 0.9 g/l, preferably approximately 0.4 g/l;
MgSO₄ From 1 to 3 g/l, preferably approximately 1.50 g/l;
Na₂SiO₃.9H₂O From 0.1 to 0.9 g/l, preferably approximately 0.5 g/l;
FeSO₄.7H₂O From 1 to 8 mg/l, preferably approximately 3 mg/l;
Na₂EDTA From 1 to 9.6 mg/l, preferably approximately 2.7 mg/l;
CaCl₂ From 0.1 to 0.25 g/l, preferably approximately 0.10 g/l;
MnCl₂.4H₂O From 1 to 5 g/l, preferably approximately 2 g/l;
CoCl₂ From 1 to 9 μg/l, preferably approximately 2.3 μg/l;
CuCl₂.2H₂O From 1 to 20 μ/l, preferably approximately 15 μg/l;
ZnCl₂ From 0.1 to 0.7 mg/l, preferably approximately 0.3 mg/l;
H₃BO₃ From 20 to 40 mg/l, preferably approximately 31.5 mg/l.

The present invention is based on observations realised by the author hereof on the occasion of the construction of a new pool for the realisation of experiments within the project giving rise to the present invention. The new pool was constructed in concrete, however for the use thereof in the experimental tests it required to be plastic-coated externally, for which purpose there was applied to the concrete a layer of paint known as gel coat and requiring dilution with acetone and cutting with paraffin. In hot climates complete curing of said paint may occur in 7 days however, in the climate of Chubut, Argentina, curing thereof was prolonged for a very much longer time. As subsequently proven, this caused the realisation of the first experimental tests carried out with active biomass under real conditions to commence without the material having fully cured and, as a consequence thereof, in the inadvertent presence of acetone in the pool and, consequently, in the composition of the culture. Under these conditions it was observed that whilst in previous experimental tests the active biomass had demonstrated rates of fixing of triglycerides in cells exceeding 14%, however, in the pool containing acetone, over the course of 10 days not only had the level of triglycerides not increased but, on the contrary, it had decreased.

Based on these observations, experiments were carried out with diverse combinations with acetone and, furthermore, with other ketones, alcohols and organic acids, some thereof yielding excellent results as inhibitors of the production of triglycerides by cells. This redounded to the benefit of the efficiency demonstrated by the biofamily by virtue of the fact that, according to what is believed, the slight but continuous stress to which the cells were subsequently subjected in the circuit of maximum production of oxygen caused therein an increase in the requirement thereof for CO₂ consumption, permitting the establishment of very long photoperiods of up to 19 hours without the cells entering a photoinhibition phase and without affecting the reproduction thereof.

Consequently, from the tests realised, it was possible to conclude that when there is present in the culture at least one organic inhibitor selected from the group comprising an alcohol, a ketone or a carboxylic acid, and in a more preferred manner ethanol, acetone or propanoic acid and/or pentanoic acid, and on then irradiating the said microorganisms with light radiation of a particular frequency, intensity and duration, by means of a mechanism which has been established although still not fully understood, the said microorganisms develop inhibition towards intra- and extracellular triglyceride fixation, leading to high metabolic activity and a requirement for carbon, this being translated into high consumption of CO₂ and emission of O₂. In a preferred embodiment, the light radiation applied to the microorganisms is multifrequency, however containing from 40 to 60%, and preferably approximately 50%, blue light having a wavelength of between 400 and 475 nm, the other wavelengths of the visible spectrum, such as red, yellow, etc, optionally excluding green, preferably participating in the remainder of the light radiation, all thereof having an intensity of at least 20 W/cm² to 38 W/cm².

Furthermore, the author hereof observed that, in the irradiation step, if the microorganisms were additionally irradiated for 3 seconds every minute at an intensity of between 5 and 15 W/cm² with ultraviolet light having a wavelength of between 400 and 200 nm without exceeding an energy of 3·10 ⁶ eV per photon, preferably in combination with the aforementioned organic inhibitor, destruction of the DMA of the cyanobacteria does not occur nor is the regime of photoinhibition thereof reached, but nevertheless the same are induced to produce up to 2.5 kg of oxygen per 2.8 kg of CO₂ provided to each kilogram of biomass.

A further characteristic of the systems and methods described, differing from other systems of the prior art, is that the biofamily of microorganisms multiplies very adequately in the described percentages at water temperatures of between 14° C. and 18° C., in comparison with other systems only operating above 22 degrees Celsius and up to 28° C. The inventor hereof has calculated that the main body of heavy industry worldwide (exceeding 60%) is located above the Tropic of Cancer, that is to say, in cold climates. Consequently, a system such as that of the present invention, being energetically balanced and achieving the purpose for which it is designed, will preferably comprise microalgae species which function optimally in cold climates.

The system of the invention is furthermore more efficient in the use of energy by virtue of the fact that it is possible to take advantage of the residual heat of the exchangers at the beginning of the system.

In order that the biofamily of microorganisms realises its function adequately, it is advantageous that suitable nutrients be made available in the culture. In a preferred embodiment, the formulation of the nutrients, arising from the study of the impact of the nitrogen cycle in nature and the different alterations thereto through human activities, is that stated below, wherein the percentages quoted are provided weekly (between 7 and 9 days) and refer to percentages by weight of the quantify of biomass resident in the system (alive):

Gaseous nitrogen (N₂): between 1% and 30%, preferably approximately 15%;
Nitric acid: between 1% and 30%, preferably approximately 7%;
Ammonium chloride (NH₄Cl): between 1% and 30%, preferably approximately 7.5%;
Phosphorus oxide (P₂O₅), from 1% to 30%;
Ammonium nitrate (NH₄NO₃): between 1% and 30%, preferably approximately 13%;
Potassium oxide (K₂O): between 1% and 40%, preferably approximately 23%;
Magnesium oxide (MgO): between 1% and 30%, preferably approximately 5%;
Sulphur trioxide (SO₃): between 1% and 40%, preferably approximately 23%;
Calcium oxide (CaO): between 1% and 50%, preferably approximately 13%;
Total boron (B): 0.05%, between 0.01% and 5%;
Total iron (Fe): 0.07%, between 0.01% and 7%;
Total zinc (Zn): 0.05%, between 0.01% and 30%;
the remainder of the culture being water, which may be fresh, brackish or salt.

In this culture the biomass has a concentration of 1 to 100 g/l, preferably approximately 27 g/l. Moreover, in the base of the fermenter tank there is preferably located a distributor promoting the dissolution of nutrients together with the dissolution and disaggregation of microscopic bubbles of CO₂, which amply facilitates absorption by the biosystem and biofamily comprising it during the artificial light photoperiod of from 14 to 18 hours. The remainder of the time, in the dark phase, atmospheric air is provided to it which, as is known, possesses a high N₂ content, which will also be present and dissolved in the water and available for the illumination phase.

4 Circuit of Maximum Production of O₂: In an optional embodiment, the culture is subsequently subjected to a step of maximum production of O₂. The underlying idea of this step of the process arose through the inventor, of Patagonian origin, identifying that the majority of the light received by the family of microorganisms utilised, originating from typically Patagonian species, was basically blue light (photosynthesis in the abyss) in the natural habitat thereof in Patagonia at a depth of 21 m and a temperature of 8° C. Nevertheless, due to the range of high and low tides, as the tide started to go down said microalgae were gradually irradiated with different light frequencies determined by the depth of the water at each moment. This, according to what is believed, has led to the microalgae biofamily utilised having become accustomed to being selective of particular light frequencies, absorbing more efficiently light at frequencies around light which is bluish. This gave rise to the idea of providing these microalgae with light irradiation of specific wavelengths around those whereto they have naturally acclimatised, although at double or greater light intensity, without substantial photoinhibition being observed therein. With this reasoning, the author of the present invention developed a photoperiod of 14 to 18 hours of artificial light having an intensity and weighted light dispersion exceeding twice that which the microorganisms would receive under natural conditions, generating exponential growth thereof, extremely high CO₂ absorption and O₂ production being a consequence thereof.

The foregoing observations have been manifested in an experimental circuit being conveniently a blind photobioreactor by virtue of the fact that the walls thereof do not require to be translucent. Consequently, it may be constructed of metal, PVC plastic or nylon, according to the conditions of location and climatology. According to the quantity of oxygen to be produced, the diameter of said tubes will be calculated for a given quantity of biomass and light radiation of irradiated light by given sector and circuit. Not all tubes require to have the same diameter by virtue of the fact that Bernoulli's principle is utilised to achieve maximum energy savings in impulsion and recirculation, together with subjecting the culture present in the circuit to the desired pressure at each moment, as a result of appropriate selection of the tubing diameter at each point. Moreover, in a particularly preferred embodiment, strips of LED diodes, optical fibre or organic LEDs will be disposed in the interior of the tube to ensure the Calvin cycle during a photoperiod irregular in light frequency and intensity. That is to say, the circuit is divided into sections wherein the biomass dwells during a given period, and in each thereof the biomass will be subjected to a particular pressure and irradiated with a particular irradiation. In agreement with a general illustrative scheme of the conditions of pressure, irradiation and period of residence of the microorganisms in each section of the module of maximum production of O₂, according to a preferred embodiment, the circuit will be divided into the following sections:
Section 1: Tubing of 25 to 100 mm in diameter. In this section the culture will be irradiated with light radiation having frequencies of between 400 and 520 nm at an irradiation intensity of between 30 and 50 W/cm² and will be subjected to a pressure of from 1.8 to 5.5 atm, remaining in the same for a period of between 10 minutes and 24 hours;
Section 2: Tubing of 63 mm to 120 mm in diameter. In this section the culture will be irradiated with light radiation having frequencies of between 521 and 580 nm at an irradiation intensity of between 10 and 20 W/cm² and will be subjected to a pressure of from 1.0 to 1.79 atm, remaining in the same for a period of between 3 minutes and 24 hours;
Section 3: Tubing of 83 mm to 180 mm in diameter. In this section the culture will be irradiated with light radiation having frequencies of between 581 and 620 nm at an irradiation intensity of between 21 and 31 W/cm² and will be subjected to a pressure of from 0.5 to 1.25 atm, remaining in the same for a period of between 3 minutes and 24 hours;
Section 4: Tubing of 181 mm to 750 mm in diameter. In this section the culture will be irradiated with light radiation having frequencies of between 621 and 750 nm at an irradiation intensity of between 30 and 5 W/cm² and will be subjected to a pressure of from 0.01 to 1.249 atm, remaining in the same for a period of between 1 minute and 24 hours.

Thus, for example, in an experimental practical embodiment, the biomass commenced being irradiated with an irradiation of between 5 W and 50 W of violet light, the biomass having a concentration of 36 g of biomass per litre of water and being displaced within the tube at a speed of between 1 km/h and 10 km/h, such that the first litre of water entering the tube returned through the other extremity 24 hours later. In this trajectory of 24 hours the different light frequencies succeeded one another, in this manner running through the light spectrum, commencing with violet light and continuing through blue, green, yellow, orange and red, to end with violet again. Between the change from one colour to another there were introduced sections of darkness wherein the microorganisms were maintained in darkness for a particular period of time, for example half an hour. In this manner the biomass will pass from a photoautotrophic state, to a heterotrophic state and to a mixotrophic state.

In a particularly preferred embodiment, through the interior of the tubes of the circuit run hoses of LEDs, organic LEDs or optical fibre maintained centred within the same by a system of double rings similar to the Mercedes Benz emblem, through the centre whereof passes a ring having bristle brushes supporting the lights, the exterior ring thereof making contact with the interior walls of the tube. All thereof are joined to one another by a thin steel cable, connected at the extremities of each tube, to an external rotary roller which receives all thereof, passing through the same number of glands preventing water from escaping. Once per month the roller, which has attached thereto a pinion and a low speed electric motor, winds the cables according to the roller of the desired extremity, causing the displacement of all rings towards the same which by means of their brushes clean the adhered biomass from both the line of lights and the exterior of the tube. In other types of photobioreactor of the prior art, after a time the surfaces thereof become saturated with biomass and manure and the efficiency thereof is reduced by up to 90%. At the end of the circuit the oxygen produced is vented.

According to a preferred embodiment, every 7 days a certain percentage of the biomass extracted is removed by cavitation to pass to the methanisation tanks. In this case new biomass is added to the fermenters in the same percentage. The entire circuit of lights preferably has a voltage of 12 V or 24 V, consuming less than 2 W per tonne of oxygen produced and which may be easily supplied by alternative energy, whether wind, photovoltaic, methane, minihydraulic, etc, if the conditions of the location so permit. In this manner a minimum energy balance is maintained for a highly efficient system.

The composition of the resultant dry biomass will depend on the characteristics of the system, on the geographical factors of the location of installation of the system, and on the multiple species utilised. A standard composition per gram of biomass having up to a maximum of 4% humidity would be the following:

### Protein: 56-71%

### Carbohydrates: 10-17%

### Lipids: 6-14%

Nucleic acids: 1-4%

### Beta-carotenes and Omegas 3, 6 and 9.

5 Biomass methanisation or deactivation tank: The biomass removed from the previous step is then optionally transferred to a tank or tanks for such purpose, which may be implemented in the form of what are known as biogas plants, having the objective of proceeding to the methanisation or deactivation of the biomass. In the equivalent systems of the prior art, to deactivate a given biomass requires a period of 10 to 12 days. On the contrary, in the present invention, by virtue of the fact that this system utilises a growth trigger for the microorganisms and more efficient and rapid deactivation/methane production, deactivation is achieved in an average of 7 days under any conditions of temperature and with any biomass. The trigger is basically organic glycerine, a very abundant and low-cost byproduct generated, for example, through the production of biodiesel. Being biodegradable it is perfectly assimilable into animal feedstuff. In this manner the biomass is deactivated preventing the methane being slowly emitted to the atmosphere, by virtue of the fact that otherwise the system would be negative in oxygen production given that 1 tonne of methane is equivalent to 21 tonnes of CO₂. If one tonne of plant or microalgae biomass produces a weighted average of 5500 litres of methane per day on adding a grade C glycerine trigger, daily production increases by 32%, however it is deactivated beforehand. That is to say that the presence of the trigger does not make the microorganisms extract more methane from a given biomass, what it does is to extract it in less time.

From this methanisation leaves a biogas which in fact is of high purity, being 30% CO₂, sent to a separator, there being obtained 30% organic CO₂ which is sent to the fermenters and 70% methane (CH₄), which is sent to an electricity generating turbine especially designed solely for use with CH₄ having a conversion efficiency exceeding 81% and very low indices of emissions, which are reinjected into the initial module of intake of gases.

In this manner, from 100% of CO₂ emissions the conversion is achieved of 40% to 60% O₂, in addition to abundant electricity from a sustainable source. This sustainable source is obtained from producing and burning the methane (CH₄), byproduct of the fermentation or deactivation of the biomass, prior to the conversion thereof into algae meal or algae flour.

6 Water treatment plant: Finally, the water having been separated from the biomass in the cavitation tank, it is optionally treated in a treatment and sterilisation plant based on the physical treatment thereof without the involvement of chemical products. Having been suitably treated it is possible to reinject if into the circuit of the fermenters.

In general, in the methods and systems of the invention natural light may be combined with artificial light in any proportion. Moreover, a fixed or variable photoperiod may be utilised, the latter option being of particular interest at sites having very marked seasons with different climates and light amplitudes. Furthermore, the fermentation tanks may optionally be installed having a particular angle with respect to the ground, for example at 45°, and having a particular orientation to achieve better solar tracking and, as a consequence, an additional increment in photosynthetic efficiency.

### EXPERIMENTAL EXAMPLES

### Example 1

In an illustrative experimental realisation, the fermenters were cylindrical tanks having a central light and therein the water for cultivation and growth of this biofamily was made to slowly rotate at 6 rpm. The culture containing the said biofamily of microorganisms had a concentration of 42 g/l of live biomass and was irradiated during a photoperiod of 14 hours per day on average with blue light of wavelength from 400 to 475 nm and with an intensity of at least 20 W/cm² to 38 W/cm². For each tonne of active biomass in the circuit there were injected thereinto, once every 30 to 45 days, the following organic inhibitors in the quantities stated:

1. From 500 ml to 7500 ml, preferably 5000 ml, of acetone;
2. From 100 ml to 5000 ml, preferably 3500 ml, of propanoic acid;
3. From 300 ml to 10 000 ml, preferably 7500 ml, of pentanoic acid;
4. From 1000 ml to 5000 ml, preferably 3000 ml, of ethanol.

As a result, the rate of growth of the biomass in all cases was greater than or equal to 8% per day of the initial quantity on installation of the circuit, whilst the total production of triglycerides calculated at the end of the experiment was only between 3 and 5% by weight with respect to the weight of the biomass. In similar experiments carried out in the absence of inhibitor the total production of triglycerides was approximately 14%, demonstrating the benefit obtained through the use of the organic inhibitor in the culture.

In a further embodiment of this practical experiment, the maximum dwell time of the biomass in the fermenter was between 4 and 6 days, a variable percentage of between 10 and 40% of said biomass being removed every 5 days on average in order to inject it into the principal absorption circuit. That is to say, the fermenters are used in this mode for the superintensive growing of biomass which may be optionally transferred to the principal absorption circuit to replace that which may be lost through mitochondrial overexcitation and profound photoinhibition. That is to say, it takes the role of what would be a seedbed in traditional agriculture.

### Example 2

In a further experimental example carried out under the same conditions as in Example 1 and with acetone as organic inhibitor, over 10 days there was obtained a total of 600 kg of biomass, there being removed a weighted average of 1.8 kg of triglycerides per kg of biomass, or 1.8%. Subsequently, the use of inhibitor was ceased, adding water to the pool at a rate of 10% per day. After 7 days samples of biomass were taken at different points of the pool until making up a total of 1 kg of extracted sample. This sample was subjected to extraction by steam distillation (selective distillation), there being obtained 8.3% of triglycerides of the total thereof compared with the previous 1.8%, thus demonstrating the efficiency of the inhibitor employed.

### Example 3: Glycerol as Trigger of the Process of Methanisation of the Biomass

Additionally, the effect was tested, in the methanisation step of the biomass, of the addition of 7% glycerol as trigger of the process. Said glycerol, dissolved in the process water itself, was added at the inlet to the methane tank. The results are shown in FIG. 4 attached, from which may be clearly deduced the multiplication effect produced by the provision of glycerol on the average production of biogas in ml/hour.

This technology is applicable to any source of emission of CO₂, although it is particularly suitable for highly contaminant sources such as cement, petrochemical, steel, petroleum and electricity generation plants, furthermore the versatility thereof permits the utilisation thereof in cities, motorways or tunnels, wherein it may capture a wide variety of gases.

1. Method for the purification of contaminated air containing CO₂ by means of microorganisms, comprising the steps of:
a) reception of air containing CO₂ proceeding from a source of contaminated air;
b) physical catalysis, wherein the air containing CO₂ is passed across plates comprising calcium and/or magnesium salts, whereon part of the CO₂ of the air becomes fixed in the form of calcium and/or magnesium carbonates;
characterised in that the method moreover comprises the steps of:
c) fermentation, wherein the air proceeding from step b) is passed across a culture comprising a biofamily of microorganisms as well as at least one organic inhibitor selected from the group consisting of an alcohol, a ketone or a carboxylic acid in any combination thereof, in which step at least part of the remaining CO₂ in the air becomes dissolved in the culture; and
d) irradiation of said culture with light radiation of the visible light spectrum having a particular frequency, intensity and duration,
such that photosynthesis is promoted in said microorganisms, causing a reduction in the CO₂ content in the culture through absorption and/or digestion of said CO₂ in the microorganisms and producing O₂.

2. Method according to claim 1, wherein the organic inhibitor of step c) is ethanol, acetone, propanoic acid or pentanoic acid in any combination thereof.

3. Method according to claim 2, wherein the organic inhibitor of step c) is acetone.

4. Method according to claim 1, wherein the step c) the biofamily of microorganisms present in the culture comprises microalgae of the classes clorphyceae, cyanophyceae, cryptophyceae, diatomaceae, and/or spores of algae being brown laminariales, red of order gigartinales or green of order ulvales, in any combination thereof.

5. Method according to claim 1, wherein the light radiation irradiated to the microorganisms in step c) is multifrequency and contains from 40 to 60% of radiation having a wavelength of between 400 and 475 nm and an intensity of between 20 W/cm² and 38 W/cm².

6. Method according to claim 5 wherein the light radiation applied to the microorganisms contains approximately 50% of radiation having a wavelength of between 400 and 475 nm and an intensity of between 20 W/cm² and 38 W/cm².

7. Method according to claim 1, wherein the light radiation irradiated to the microorganisms in step c) consists substantially of radiation having a wavelength of between 400 and 475 nm and an intensity of between 20 W/cm² and 38 W/cm².

8. Method according to claim 5, wherein in step c) for 3 seconds every minute the microorganisms are furthermore irradiated with additional light radiation having a wavelength of approximately 200 nm and an intensity of between 5 and 15 W/cm², without exceeding an energy of 3·10⁶ eV per photon.

9. Method according to claim 1, wherein the dwell time of the microorganisms in step c) is from 4 to 6 days, and every 5 days there is removed therefrom a percentage varying from 10% to 40% of the microorganisms.

10. Method according to claim 1, wherein following step d), the following additional step is applied:
e) maximum production step of O₂, wherein the culture from step d) is passed along a circuit wherein it is subjected to a series of pressures in succession and to irradiation with a series of frequencies of the light spectrum also in succession, thereby causing an additional reduction in the CO₂ content in the culture through absorption and/or digestion of said CO₂ in the microorganisms and producing O₂.

11. Method according to claim 10, wherein the step e), the sequence of pressures to which the culture is subjected lies between 0.01 atm and 5.5 atm, and the sequence of frequencies of the light spectrum to which the culture is subjected passes through violet, blue, green, yellow, orange and red, returning to violet, the radiation having an intensity of between 5 and 50 W/cm².

12. Method according to claim 11, wherein the culture moreover is subjected to the following sequence of light radiation:
a first section wherein the culture is irradiated with light radiation having frequencies of between 400 and 520 nm at an irradiation intensity of between 30 and 50 W/cm² and is subjected to a pressure of from 1.8 to 5.5 atm, remaining in the same for a period of between 10 minutes and 24 hours;
a second section wherein the culture is irradiated with light radiation having frequencies of between 521 and 580 nm at an irradiation intensity of between 10 and 20 W/cm² and is subjected to a pressure of from 1.0 to 1.79 atm, remaining in the same for a period of between 3 minutes and 24 hours;
a third section wherein the culture is irradiated with light radiation having frequencies of between 581 and 620 nm at an irradiation intensity of between 21 and 31 W/cm² and is subjected to a pressure of from 0.5 to 1.25 atm, remaining in the same for a period of between 3 minutes and 24 hours;
a fourth section wherein the culture is irradiated with light radiation having frequencies of between 621 and 750 nm at an irradiation intensity of between 30 and 5 W/cm² and is subjected to a pressure of from 0.01 to 1.249 atm, remaining in the same for a period of between 1 minute and 24 hours.

13. Method according to claim 12, wherein between each section and that following the culture is subjected to a period of darkness wherein it is not irradiated.

14. Method according to any of foregoing claim 12 wherein the sequence of irradiations is applied to the culture in photoperiods of from 14 to 18 hours per day.

15. Method according to claim 1, wherein every particular number of days at least a part of the microorganisms is removed from the circuit of maximum production of O₂ and transferred to an additional step of:
f) methanisation, wherein the microorganisms are deactivated by means of a procedure of fermentative oxidation, there being obtained a deactivated biomass mixed with water and a biogas comprising CO₂ and methane.

16. Method according to claim 15, wherein fermentative oxidation is carried out utilising an inducer accelerating the process, said inducer being glycerol.

17. Method according to claim 15, wherein the water obtained in the procedure of fermentative oxidation is sterilised and returned to the circuit at the fermentation step.

18. System to purify contaminated air containing CO₂ by means of microorganisms comprising the following elements:
a) reception systems (1) receiving air containing CO₂ proceeding from a source of contaminated air;
b) system of plates (2) containing calcium and/or magnesium salts intended to fix part of the CO₂ of the air in the form of calcium and/or magnesium carbonates;
characterised in that the method further comprises:
c) tanks (3) for fermentation of the air proceeding from the previous step wherein is included a culture comprising a family of microorganisms as well as at least one organic inhibitor selected from the group consisting of an alcohol, a ketone or a carboxylic acid in any combination thereof,
wherein such tanks further comprise a source of light radiation intended to irradiate the culture with light radiation of the visible light spectrum having a particular frequency, intensity and duration.

19. System according to claim 18, wherein the biofamily of microorganisms present in the culture contained in fermentation tanks (3) comprises microalgae of the classes clorophyceae, cyanophyceae, cryptophceae, diatomaceae, and/or spores of algae being brown laminariales, red of order gigartinales or green of order ulvales, in any combination thereof.

20. System according to claim 18, wherein the organic inhibitor contained in the culture present in fermentation tanks (3) is ethanol, acetone, propanoic acid or pentanoic acid, in any combination thereof.

21. System according to claim 20, wherein the organic inhibitor is acetone.

22. System according to any of claim 18, furthermore comprising circuit (4) of maximum production of O₂, including an assembly of tubing intended for the passage therethrough of the culture from fermentation tanks (3), which tubing comprises means capable of subjecting the culture to a particular sequence of pressures and a source of light radiation capable of irradiating the culture with a particular sequence of frequencies of the light spectrum, simultaneously.

23. System according to claim 22, wherein the pressure means capable of subjecting the culture to a particular sequence of pressures comprise sections of tubing of different diameters.

24. System according to claim 23, wherein the tubing has a diameter of between 25 and 750 mm.

25. System according to claim 18, wherein the pressure means are capable of subjecting the culture to a sequence of pressures of between 0.01 atm and 5.5 atm and the source of light radiation is capable of irradiating the culture with a sequence of frequencies of the light spectrum passing through violet, blue, green, yellow, orange and red, returning again to violet, with an irradiation intensity of between 5 and 50 W/cm².

26. System according to claim 18, further comprising methanisation tanks (5) intended to deactivate the microorganisms by means of a procedure of fermentative oxidation, there being obtained therefrom a deactivated biomass mixed with water and a biogas comprising CO₂ and methane.

27. System according to claim 26, further comprising a turbine (6) intended to obtain electricity from the methane obtained in the methanisation tanks (5).

28. System according to claim 27, further comprising a sterilisation system (7) for sterilising the water obtained in the methanisation tanks (5).
